(19) United States
(12) Patent Application Publication
(10) Pub. No.: US 2006/0073216 A1
(43) Pub. Date: Apr. 6, 2006

(54) Compositions and methods for control of bovine mastitis

(75) Inventor: James L McNaughton, Quantico, MD (US)

Correspondence Address: SIEBERTH & PATTY, LLC, 4703 BLUEBONNET BLVD, BATON ROUGE, LA, 70809, US

(73) Assignee: Solution Biosciences, Inc.

(21) Appl. No.: 10/540,378

(22) PCT Filed: Dec. 26, 2002
(86) PCT No.: PCT/US02/41479

§ 371 Date: Jun. 23, 2005

(51) Int. Cl. A61K 33/14; A61K 31/4166

(52) U.S. Cl. 424/661; 514/389; 424/723

(57) ABSTRACT

Bovine mastitis is prevented or controlled by treating at least teats of the animal with an effective antimicrobial amount of A) an aqueous microbiocidal solution of one or more active halogen species, which solution is a derivative product in an aqueous medium of (i) bromine, chlorine, and bromine chloride, or any two or all three thereof, and (ii) a watersoluble source of sulfamate anion; or B) an aqueous microbiocidal solution of one or more active halogen species, which solution is a derivative product in an aqueous medium of at least one 1,3-dihalo-5,5-dialkylhydantoin in which one of the halogen atoms is a bromine atom and the other halogen atom is a chlorine or bromine atom, and in which when both halogen atoms are bromine atoms, one of the alkyl groups is a methyl group and the other alkyl group contains in the range of 1 to about 4 carbon atoms, and when one of the halogen atoms is a bromine atom and the other halogen atom is a chlorine atom, the alkyl groups, independently, each contain in the range of 1 to about 4 carbon atoms; or C) an aqueous microbiocidal solution of A) and an aqueous microbiocidal solution of B).

### BACKGROUND

A highly important factor in the management of dairy farms is preventing or at least maitaining very effective control of bovine mastitis in the herd. As is well known in the art, there are in general two types of bovine mastitis infections, namely contagious and environmental. Contagious mastitis can be transmitted and spread during the milking process through contact of the animal with milking apparatus which may carry a source of mastitis pathogen. Environmental mastitis can be caused by contamination of the animal skin by materials with which the animal comes in contact as it moves through its environment, such as barns, barnyards and fields. In the case of environmental mastitis, the infection can occur not only when the animal is producing milk but also during the “dry” or “non-lactating” period, i.e., the period of weeks immediately preceding the delivery of a calf during which milk production in the animal temporarily ceases.

To prevent or control bovine mastitis infection, two general approaches have been used. One involves application of substances that form water-soluble films on the teats including portions of the teat canal exterior to the sphincter and the surrounding udder area. Such films, which are washed off before milking, are intended to provide barrier protection against contamination by pathogens between milkings. In the absence of an antimicrobial agent (a.k.a germicide or microbiocide), the effectiveness of this approach depends entirely upon the integrity of the film, and thus some film-forming formulations also include an antimicrobial agent. The other approach involves contacting the teat and surrounding udder area with an antimicrobial agent, which typically is applied by spraying or dipping after completion of the milking.

For a recent fairly extensive bibliography of disclosures relating to technology relating to the general approaches used for control of bovine mastitis, see U.S. Pat. No. 6,436,444, especially the passages from Column 2, line 55 through Column 3, line 44. Also given in that patent is a list of commercial teat dips of varying degrees of effectiveness in which the antimicrobial agents employed listed are iodophors, quaternary ammonium compounds, chlorhexidine salts, chlorine release compounds (e.g., alkali hypochlorites), oxidizing compounds (e.g., hydrogen peroxide, peracids), protonated carboxylic acids (e.g, heptanoic, octanoic, nonanoic, decanoic, undecanoic acids), acid anionics (e.g., alkylaryl sulfonic acids), and chlorine dioxide (from chlorite).

Although halogen-based antimicrobial agents most often referred to in connection with prevention or control of bovine mastitis are based on iodine or chlorine, there have been some references to bromine-containing antimicrobial agents for this use. Thus each of U.S. Pat. Nos. 4,199,602; 4,258,056; and 4,376,787 mentions that bromine itself had been previously investigated, and U.S. Pat. No. 4,258,056, in referring to nitroalkanols as a component of the teat dip formulations described therein, indicates a preference for 2-bromo-2-nitropropane-1,3-diol, in Table 2 provides comparative test data for this and various proprietary teat dip compounds. This specific compound (identified as bronopol) is also mentioned as a member of a group of antimicrobial agents that may be used in the teat dip formulations therein described. U.S. Pat. No. 5,017,369 also indicates that prior art had suggested use of bromine for use against bovine mastitis. And U.S. Pat. Nos. 6,379,685 and 6,436,444 refer, among other things, to use of alkali and alkaline earth hypobromites as bromine release agents for use in teat dip compositions.

### SUMMARY OF THE INVENTION

This invention provides new compositions and methods enabling highly effective control or prevention of bovine mastitis. High antimicrobial effectiveness may be achieved even when the contact times used are relatively short. Also, because of the high microbiocidal effectiveness of the halogen-containing microbiocides used in this invention, especially the bromine-containing microbiocides used in this invention, it is possible to readily produce and use aqueous treating formulations (e.g., teat dip and spray compositions) having low concentrations of the microbiocidal agent. This in turn reduces the possibility of irritation to teat tissues and surrounding skin surfaces of the udder.

In one of its embodiments, this invention provides a method of preventing or controlling bovine mastitis, which method comprises treating at least the teats of the animal with an effective antimicrobial amount of:
A) a composition comprised of an aqueous microbiocidal solution of one or more active halogen species, which solution is a derivative product in an aqueous medium of (i) bromine, chlorine, and bromine chloride, or any two or all three thereof, and (ii) a water-soluble source of sulfamate anion; or
B) a composition comprised of an aqueous microbiocidal solution of one or more active halogen species, which solution is a derivative product in an aqueous medium of at least one 1,3-dihalo-5,5-dialkylhydantoin in which one of the halogen atoms is a bromine atom and the other halogen atom is a chlorine or bromine atom, and in which when both halogen atoms are bromine atoms, one of the alkyl groups is a methyl group and the other alkyl group contains in the range of 1 to about 4 carbon atoms, and when one of the halogen atoms is a bromine atom and the other halogen atom is a chlorine atom, the alkyl groups, independently, each contain in the range of 1 to about 4 carbon atoms; or
C) a composition comprised of an aqueous microbiocidal solution of A) and an aqueous microbiocidal solution of B), as separate solutions or preferably as a single solution.

In another of its embodiments, this invention provides a composition suitable for preventing or controlling bovine mastitis, which composition comprises:
A) an aqueous microbiocidal solution of one or more active halogen species, which solution is a derivative product in an aqueous medium of (i) bromine, chlorine, and bromine chloride, or any two or all three thereof, and (ii) a water-soluble source of sulfamate anion; or
B) an aqueous microbiocidal solution of one or more active halogen species, which solution is a derivative product in an aqueous medium of at least one 1,3-dihalo-5,5-dialkylhydantoin in which one of the halogen atoms is a bromine atom and the other halogen atom is a chlorine or bromine atom, and in which when both halogen atoms are bromine atoms, one of the alkyl groups is a methyl group and the other alkyl group contains in the range of 1 to about 4 carbon atoms, and when one of the halogen atoms is a bromine atom and the other halogen atom is a chlorine atom, the alkyl groups, independently, each contain in the range of 1 to about 4 carbon atoms; or
C) an aqueous microbiocidal solution of both A) and B); wherein the composition additionally contains at least one of the following components:
D) at least one thickener, or
E) at least one water-soluble polymeric film-forming agent; or
F) at least one emollient or humectant.
   As regards components D), E), and F), preferred compositions contain both of D) and F), or both of E) and F), and particularly preferred compositions contain each of D), E), and F).

Another embodiment is the provision and use of two separate microbiocidal solutions, one such composition being comprised of A) and at least one of D), E), and F), and preferably both of D) and F), or both of E) and F), and more preferably all three of D), E), and F); and the other such composition being B). Preferably these two separate solutions can be provided in separate suitably labeled containers as a kit with appropriate instructions for use. The two solutions can be used consecutively in either order, or they can be used alternately, e.g., one such composition can be used after one milking and the other composition after the ensuing milking, and so on.

Still another embodiment is the provision and use of two separate microbiocidal solutions, one such composition being comprised of B) and at least one of D), E), and F), and preferably both of D) and F), or both of E) and F), and more preferably all three of D), E), and F); and the other such composition being A). Preferably these two separate solutions can be provided in separate suitably labeled containers as a kit with appropriate instructions for use. The two solutions can be used consecutively in either order, or they can be used alternately, e.g., one such composition can be used after one milking and the other composition after the ensuing milking, and so on.

Yet another embodiment is the provision and use of two separate microbiocidal solutions, one such composition being comprised of A) and at least one of D), E), and F), and preferably both of D) and F), or both of E) and F), and more preferably all three of D), E), and F); and the other such composition being B) and at least one of D), E), and F), and preferably both of D) and F), or both of E) and F), and more preferably all three of D), E), and F). Preferably these two separate solutions can be provided in separate suitably labeled containers as a kit with appropriate instructions for use. The two solutions can be used consecutively in either order, or they can be used alternately, e.g., one such composition can be used after one milking and the other composition after the ensuing milking, and so on.

In all of the above embodiments, the one or more active halogen species of the microbiocidal solutions is preferably one or more of the above active bromine species, since the active bromine species are more effective than the corresponding active chlorine species. Most preferred are active bromine species resulting from dissolving a 1,3-dibromo-5,5-dialkylhydantoin as described above in an aqueous medium.

Other embodiments and features of this invention will be still further apparent from the ensuing description and appended claims.

### FURTHER DETAILED DESCRIPTION OF THE INVENTION

The derivative product of A) above is an aqueous microbiocidal solution of one or more active halogen species, which solution is formed by and thus results from a reaction in water between bromine, chlorine, or bromine chloride, or any two or all three thereof, and a water-soluble source of sulfamate anion. A concentrated solution of this type containing over 100,000 ppm of active halogen is available commercially from Albemarle Corporation under the trademark STABROM ® 909 biocide. Because of its enhanced stability, a concentrated solution such as this can be stored under ambient room temperature conditions for suitably long periods of time before use. As described more fully hereinafter, if such a concentrated solution is to be used, it must be suitably diluted with water to an appropriate antimicrobial concentration. Also again as described more fully hereinafter, because such concentrated solutions as supplied have a high pH typically a pH of 13 or more, the concentrated solution should be treated with a suitable acidic substance to reduce its pH to a suitable level before application to the animal.

Purely for convenience, the microbiocides of A) described above when made from bromine chloride, bromine and chlorine, or bromine, chlorine, and bromine chloride, and a sulfamate source, are sometimes referred to hereinafter as “sulfamate-stabilized bromine chloride” even though technically the actual chemical species in the aqueous medium are most probably not bromine chloride molecules or sulfamate adducts or complexes of bromine chloride. Thus the designation “sulfamate-stabilized bromine chloride” is simply a shorthand way of referring to such compositions, and the designation does not signify, suggest, or imply anything about the actual chemical structure of the composition.

The halogen-based microbiocides of B) above are microbiocidal solutions of one or more active halogen species including or consisting of active bromine species, which solutions are derivative products in an aqueous medium such as water of at least one 1,3-dihalo-5,5-dialkylhydantoin in which one of the halogen atoms is a bromine atom and the other is a chlorine or bromine atom and the alkyls are as described above. Upon dissolving in an aqueous medium such as water a 1,3-dihalo-5,5-dialkylhydantoin referred to in this paragraph, a transformation takes place so that active halogen or active bromine species are present in the resultant solution.

In preferred embodiments, the halogen-based microbiocide used in the practice of this invention is a bromine-based microbiocide comprising an over based aqueous microbiocidal solution of one or more active bromine species, said species resulting from a) a reaction in water between bromine or bromine chloride, a mixture of bromine chloride and bromine, or a combination of bromine and chlorine in which the molar amount of chlorine is either equivalent to the molar amount of bromaine or less than the molar amount of bromine, and a water-soluble source of sulfamate anion, or b) an aqueous microbiocidal solution of at least one 1,3-dibromo-5,5-alkylhydantoin in which one of the alkyl groups is a methyl group and the other alkyl group contains in the range of 1 to about 4 carbon atoms, or c) both of a) and b) hereof. Thus for convenience, the term “bromine-based” means any of the microbiocides referred to in this paragraph as a), b), or c).

The bromine-based microbiocides used in this invention are more effective than the corresponding chlorine-based microbiocides against various bacteria both gram-positive bacteria and gram-negative bacteria, and including coliform bacteria In addition, these bromine-based microbiocides tend to be less odorous than chlorine-based microbiocides, and are essentially devoid of unwanted bleaching activity. Moreover, while some of the bromine-based microbiocides may possibly react with nitrogenous species, such as may be present on the teats and/or udder surfaces due to contact with coliform contaminated ground surfaces or the like, the resultant bromides formed in situ by reaction with the treating agent would also possess microbiological activity. Thus such side reactions would not materially decrease the microbiological effectiveness made available to the dairy farmer by use of these bromine-based microbiocides. Furthermore, bromamines would not exhibit obnoxious properties toward workers in the barn or other milking areas whereas chloramines which can result from use of certain chlorine-based microbiocides under the same conditions tend to be powerful lachrymators.

As noted above, the halogen-based microbiocides of A) above are microbiocidal solutions of one or more active halogen species, which solutions are derivative products in a aqueous medium such as water of bromine, chlorine, or bromine chloride, or any two or all three thereof, and a water-soluble source of sulfamate anion. Likewise, the preferred bromine-based microbiocides of a) above are microbiocidal solutions of one or more active bromine species, which solutions are derivative products in a aqueous medium such as water of bromine or bromine chloride, a mixture of bromine chloride and bromine, or a combination of bromine and chlorine in which the molar amount of chlorine is either equivalent to the molar amount of bromine or less than the molar amount of bromine, and a water-soluble source of sulfamate anion. To form these derivative products the components from which the derivative products are formed are brought together in an aqueous medium such as water, which medium or water, when forming the product, preferably is always at a pH of at least 7 and more preferably is always at a pH higher than 7, e.g., in the range of 10-14, by use of an inorganic base such as sodium hydroxide. When using a commercially-available product of this type (Stabrom® 909 biocide; Albemarle Corporation), the pH of the aqueous product as received is normally in the range of 13 to 14.

The aqueous microbiocidal solutions used pursuant to this invention can be formed by mixing a preformed concentrated aqueous solution of the microbiocidal agent (i.e., in undiluted form) with water to form a suitably dilute treating solution for use with the animal. Alternatively, the 1,3-dihalo-5,5-dialkylhydantoin itself can be added to and mixed with water to form a suitably dilute treating solution for use with the animal. The solubility of 1,3-dibromo-5,5dimethylhydantoin in water at 75° F. (ca. 24° C.) is about 405 ppm expressed as Cl₂ whereas the solubilities of N,N′-bromochloro-5,5-dimethylhydantoin and of the commercial mixture of N,N′-bromochloro-5,5-dimethylhydantoin and 1,3-dichloro-5-ethyl-5-methylhy-dantoin at the same temperature are, respectively, about 890 ppm and 1905 ppm, both expressed as Cl₂. The contacting of the suitably dilute aqueous microbiocidal solution used pursuant to this invention however produced and applied to the teats and surrounding udder areas results in effective protection against bovine mastitis. Contact times can be as short as 5 seconds or less, but can be longer if deemed necessary. A contact time of not more than about 15 seconds is especially preferred.

At present the most preferred bromine-based microbiocide used in the practice of any embodiment of this invention is a water-soluble 1,3-dibromo-5,5-dialkylhydantoin in which one of the alkyl groups is a methyl group and the other is an alkyl group containing from 1 to about 4 carbon atoms, with 1,3-dibromo-5,5-dimethylhydantoin being the most preferred of all.

In practice, the teats and preferably at least a portion of surrounding udder area to be disinfected are contacted with the aqueous microbiocidal solutions referred to above which of course contain a microbiocidally-effective amount of the microbiocidal agent and/or microbiocidal hydrolysis product(s) thereof. The contacting can be effected by use of sprays, teat dips, or other apparatus designed to bring the antimicrobial solution into contact with the area to be disinfected. Usually this operation will be performed after recovering the milk from the animal. However the operation can be conducted prior to milking, but in this case use a thorough washing of the treated surfaces with clean water before milking is recommended.

One group of halogen-based microbiocides for use in the practice of this invention is an aqueous microbiocidal solution of one or more active halogen species of type A) above, said species resulting from a reaction in water between bromine, chlorine, or bromine chloride, or any two or all three thereof, and a water-soluble source of sulfamate anion. If sulfamic acid is used in forming this microbiocide, the solution should also be provided with a base, preferably enough base to keep the solution alkaline, ie., with a pH above 7, preferably above about 10 and most preferably about 13 or above. The lower the pH, the more unstable the solution, and thus if the solution is prepared on site for immediate use, the use of a base is not essential. However, it is preferable to employ a concentrated microbiocidal solution manufactured elsewhere, and in such case the concentrated solution would be provided as an overbased solution with a pH of, say, about 13 or more. Often such concentrated solutions will contain over 50,000 ppm (wt/wt) of active halogen, preferably at least about 100,000 ppm (wt/wt) of active halogen, and sometimes as much as about 150,000 ppm (wt/wt) or more of active halogen, active halogen content being determinable by use of conventional starch-iodine titration.

One preferred group of type A) above is a bromine-based microbiocidal solution formed by reacting bromine or, more preferably bromine chloride, a mixture of bromine chloride and bromine, or a combination of bromine and chlorine in which the molar amount of chlorine is either equivalent to the molar amount of bromine or less than the molar amount of bromine, in an aqueous medium with sulfamic acid and/or a water-soluble salt of sulfamic acid. Except when made on site for immediate use, such solutions should be highly alkaline solutions typically with a pH of at least about 12 and preferably at least about 13, such pH resulting from use of a base such as sodium hydroxide or the like, in producing the solution. Concentrated solutions of this type are available in the market place, for example, Stabrom® 909 biocide (Albemarle Corporation). Processes for producing these concentrated aqueous microbiocidal solutions are described in U.S. Pat. Nos. 6,068,861, issued May 30, 2000, and 6,299,909, issued Oct. 9, 2001. Because the Stabrom® 909 biocide solution as supplied has a high pH, before use on the animal, the portion of the concentrated solution to be used should be treated with an acidic substance (e.g. a mineral acid such as HCl, H₂SO₄, H₃PO₄, or H₃PO₃, etc., or a water-soluble organic acid such as formic acid, acetic acid, propionic acid, d-tartaric acid, or mesotartaric acid, etc. to reduce the pH of the solution to a pH in the range of about 5 to about 9 and also with water to form a dilute solution containing an antimicrobial amount of active bromine in the range of about 15 to about 200 ppm (wt/wt) of active bromine, and preferably in the range of about 50 to about 150 ppm (wt/wt) of active bromine as determinable by the DPD test procedure.

It will be appreciated that even where the microbiocide is made from bromine chloride, a mixture of bromine chloride and bromine, or a combination of bromine and chlorine in which the molar amount of chlorine is either equivalent to the molar amount of bromine or less than the molar amount of bromine is used, the microbiocide is bromine-based as most of the chlorine usually winds up as a chloride salt such as sodium chloride since an alkali metal base such as sodium hydroxide is typically used in the processing to raise the pH of the product solution to at least about 13. Thus the chlorine in the product solution is not present as a significant microbiocide.

Another group of halogen-based microbiocides for use in the practice of this invention is of type B) above, i.e., one or more N,N′-halo-5,5-dialkylhydantoins in which one of the halogen atoms is bromine and the other is chlorine, and in which the alkyl groups, independently, each contain from 1 to about 4 carbon atoms. Suitable compounds of this type include, for example, such compounds as N,N′-bromochloro-5,5-diimethylhydantoin, N,N′-bromochloro-5-ethyl-5-methylhy-dantoin, N,N-bromochloro-5-propyl-5-methylhydantoin, N,N′-bromochloro-5-isopropyl-5-methylhydantoin, N,N′-bromochloro-5-butyl-5-methylhydantoin, N,N′-bromochloro-5-isobutyl-5-methylhydantoin,N,N′-bromochloro-5-sec-butyl-5-methylhydantoin, N,N′-bromochloro-5-tert-butyl-5-methylhydantoin, N,N′-bromochloro-5,5-diethylhydantoin,and mixtures of any two or more of the foregoing. N,N′-bromochloro-5,5-dimethylhydantoin is available commercially under the trade designation Bromicide® biocide (Great Lakes Chemical Corporation). Another suitable bromochlorohydantoin mixture is composed predominantly of N,N′-bromochloro-5,5-dimethylhydantoin together with a minor proportion by weight of 1,3-dichloro-5-ethyl-5-methylhydantoin. A mixture of this latter type is available in the marketplace under the trade designation Dantobrom® biocide (Lonza Corporation).

When a mixture of two or more of the foregoing N,N-bromochloro-5,5-dialkylhydantoin biocides is used pursuant to this invention, the individual biocides of the mixture can be in any proportions relative to each other.

It will be understood that the designation N,N′ in reference to, say, N,N″-bromochloro-5,5-dimethylhydantoin means that this compound can be (1) 1-bromo-3-chloro-5,5dimethylhydantoin, or(2) 1-chloro-3-bromo-5,5-dimethylhydantoin,or (3) a mixture of 1-bromo-3-chloro-5,5-dimethylhydantoin and 1-chloro-3-bromo-5,5-dimethylhydantoin. Also, it is conceivable that some 1,3-dichloro-5,5-dimethylhydantoin and 1,3-dibromo-5,5-dimethylhydantoin could be present in admixture with (1), (2), or (3).

A preferred system for use in the practice of this invention is a bromine-based microbiocidal solution of a 1,3-dibromo-5,5-dialkylhydantoin in which one of the alkyl groups is a methyl group and the other alkyl group contains in the range of 1 to about 4 carbon atoms. Thus these preferred biocides comprise 1,3-dibromo-5,5-dimethylhydantoin, 1,3-dibromo-5-ethyl-5-methylhydantoin, 1,3-dibromo-5-n-propyl-5-methylhydantoin, 1,3-dibromo-5-isopropyl-5-methyl-hydantoin, 1,3-bromo-5-n-butyl-5-methylhydantoin, 1,3-dibromo-5-isobutyl-5-methylhydantoin, 1,3-dibromo-5-sec-butyl-5methylhydantoin, 1,3-dibromo-5-tert-butyl-5-methylhydantoin, and mixtures of any two or more of them. Of these biocidal agents, 1,3-dibromo-5-isobutyl-5-methylhydantoin, 1,3dibromo-5-n-propyl-5-methylhydantoin, and 1,3-dibromo-5-ethyl-5-methylhydantoin are, respectively, preferred, more preferred, and even more preferred members of this group from the cost effectiveness standpoint. Of the mixtures of the foregoing biocides that can be used pursuant to this invention,it is preferred to use 1,3-dibromo-5,5-dimethylhydantoin as one of the components, with a mixture of 1,3-bromo-5,5dietlylhydantoin and 1,3-dibromo-5-ethyl-5-methylhydantoin being particularly preferred. The most preferred member of this group of microbiocides is 1,3-dibromo-5,5-dimethylhydantoin. This compound is available in the marketplace in under the trademark XtraBrom™ 111 biocide (Albemarle Corporation).

When a mixture of two or more of the foregoing 1,3-dibromo-5,5-dialkylhydantoin biocides is used pursuant to this invention, the individual biocides of the mixture can be in any proportions relative to each other. Methods for producing 1,3-dibromo-5,5-dialkylhydantoins are known and reported in the literature. One such efficacious process is described in WO 01/53270, published Jul. 26, 2001.

If desired, the 1,3-dihalo-5,5-dialkylhydantoins can be dissolved in a suitable innocuous, harmless, inert, water-soluble organic solvent with or without water to form either a suitably dilute solution for use or a concentrated solution for dilution before use. Care should be taken to ensure that such organic solvent is resistant to the oxidizing effect of the 1,3-dihalo-5,5dialkylhydantoin, and that it will cause no harm to the tissues of the animal during use. If desired, the treated surfaces of the animal can then be further washed with clean water to remove residues from such solvent. Besides increasing the amount of 1,3-dihalo-5,5-dialkylhydantoin that can be put into solution thus facilitating formation of a concentrated solution, e.g., on the premises of the milking operation, such a concentrated solution when diluted such by addition to water being used on the premises possesses microbiocidal activity from the 1,3-dihalo-5,5-dialkylhydantoin. Thus aqueous solutions used pursuant to this invention can contain suitably small amounts of an innocuous, harmless, water-soluble oxidation resistant organic solvent, which is non-toxic, at least at the dosage levels involved.

In cases where extremely power biocidal activity is desired such as during periodic cleaning and disinfection of milking apparatus, milk containers, pasteurizing apparatus, etc., concentrated aqueous solutions of the microbiocides of this invention can be directly applied to the surfaces of such apparatus or equipment to protect against infestation with pathogenic microorganisms. Such concentrated solutions can contain, for example, as much as 150,000 ppm or 160,000 ppm or more of active bromine, and as much as about 66,667 ppm or about 71,111 ppm of active chlorine, as determinable by conventional starch-iodine titration. If desired, a portion of such concentrated solution can be diluted with any suitable amount of water before application directly to the surfaces of such apparatus or equipment, provided of course that the diluted solution still contains a microbiocidally-effective amount of active bromine species for the use at hand. Also, concentrated solutions of this invention can be added to and thus used in diluted form in process water being used in milk processing operations, such as for example, in water flowing through conduits, in water flowing into or being maintained in tanks, and in water being used in spraying equipment. Thus concentrated solutions of types A) and B) above can serve both as sources of suitably dilute antimicrobial solutions for application to the teats and surrounding udder areas of the cows and also as sources of antibacterial solutions of varying strengths for use in sanitizing apparatus and equipment present in the milk producing facility, thus minimizing inventory requirements.

The amount (concentration) of the selected microbiocide utilized in the practice of this invention will vary depending on various factors such as the particular microbiocide being used, the interval between microbiocidal treatments, the types and nature of the microorganisms present, the amount and types of nutrients available to the microorganisms, and so on. In any event, a microbiocidally-effective amount of the diluted aqueous solution of the microbiocide used pursuant to this invention will be applied to or contacted with the teat and surrounding udder surfaces, such as by dipping or spraying, or both. Typically the diluted solution will contain a microbiocidally-effective amount of active halogen in the range of about 15 to about 200 ppm (wt/wt), preferably in the range of about 50 to about 150 ppm (wt/wt), and more preferably in the range of about 75 to about 100 ppm (wt/wt), active halogen being determinable by use of the conventional DPD test procedure. If the actual active halogen in the solution consists of active chlorine from a type A) microbiocidal solution in a case where chlorine is used as the sole halogen source, the concentration of the diluted solution used is preferably at least two to three times higher than the minimums of the foregoing ranges. In the case of the 1,3-dibromo-5,5-diaIkylhydantoin solutions used pursuant to this invention in preventing or controlling bovine mastitis, a preferred concentration for use is typically within the range of about 15 to about 200 ppm (wt/wt) and more preferably in the range of about 50 to about 150 ppm (wt/wt) of active bromine as determinable by the DPD test procedure. Similar concentration ranges are applicable when using sulfamate-stabilized bromine chloride in the treatment of the animals pursuant to this invention. Contact times are typically in the range of up to about 3 minutes, and preferably are in the range of about 5 seconds or less up to about 2 minutes. The concentration and contact time should of course be such that the treated portions of the animal are not adversely affected or that the animal is otherwise distressed.

As can be seen from the above, there are two different types of procedures that are used for determining active halogen content, whether active chlorine, active bromine or both. For measuring concentrations in the vicinity of above about, say, 500 ppm or so (wt/wt) of active bromine or, say, above about 1100 ppm of active chlorine, starch-iodine titration is the preferred procedure. On the other hand, where concentrations are below levels in these vicinities,the conventional DPD test procedure is more suitable, as this test is designed for measuring very low active halogen concentrations, e.g., active chlorine concentrations in the range of from zero to about 11-12 ppm (wt/wt) or active bromine concentrations in the range of from zero to about 5 ppm (wt/wt). In fact, where the actual concentration of active chlorine is between, say, about 11-12 ppm and about 1100 ppm (wt/wt), or the where the actual concentration of active bromine is between, say, about 5 ppm and about 1100 ppm (wt/wt), the test sample is typically diluted with pure water to reduce the actual concentration to be in the range of about 4 to about 11-12 ppm in the case of active chlorine and to be in the range of about 2 to about 5 ppm in the case of active bromine before making the DPD analysis. It can be seen therefore that while there is no critical hard-and-fast concentration dividing line between which procedure to use, the approximate values given above represent a practical approximate dividing line, since the amounts of water dilution of more concentrated solutions when using the DPD test procedure increase with increasing initial active halogen concentration, and such large dilutions can readily be avoided by use of starch-iodine titration when analyzing the more concentrated solutions. In short, with suitably dilute solutions use of the DPD test procedure is recommended, and with more concentrated solutions use of starch-iodine titration is recommended.

The starch-iodine titration procedure for determination of active halogen has long been known. For example, chapter XIV of Willard-Furman, Elementary Quantitative Analysis, Third Edition, D. Van Nostrand Company,Inc., New York, Copyright 1933, 1935, 1940 provides a description of starch-iodine titration. While details of standard quantitative analytical procedures for determination of active halogen in such product solutions by starch-iodine titration may vary from case to case, the results are normally sufficiently uniform from one standard procedure to another as not to raise any question of unreliability of the results. A recommended starch-iodine titration procedure is as follows: A magnetic stirrer and 50 milliliters of glacial acetic acid are placed in an iodine flask. The sample (usually about 0.2-0.5 g) for which the active halogen is to be determined is weighed and added to the flask containing the acetic acid. Water (50 milliliters) and aqueous potassium iodide (15%, wt/wt; 25 milliliters) are then added to the flask The flask is stoppered using a water seal. The solution is then stirred for fifteen minutes, after which the flask is unstoppered and the stopper and seal area are rinsed into the flask with water. An automatic buret (Metrohm Limited) is filled with 0.1 normal sodium thiosulfate. The solution in the iodine flask is titrated with the 0.1 normal sodium thiosulfate; when a faint yellow color is observed, one millimeter of a 1 wt % starch solution in water is added, changing the color of the solution in the flask from faint yellow to blue. Titration with sodium thiosulfate continues until the blue color disappears. The amount of active halogen is calculated using the weight of the sample and the volume of sodium thiosulfate solution titrated. In this way, the amount of active halogen such as active chlorine or active bromine in an aqueous product solution, regardless of actual chemical form, can be quantitatively determined.

The standard DPD test for determination of low levels of active halogen is based on classical test procedures devised by Palinin 1974. See A. T. Palin, “Analytical Control of Water Disinfection With Special Reference to Differential DPD Methods For Chlorine, Chlorine Dioxide, Bromine, Iodine and Ozone”, J Inst. Water Eng., 1974, 28, 139. While there are various modernized versions of the Palin procedures, the recommended version of the test is filly described in Hach Water Analysis Handbook, 3rd edition, copyright 1997. The procedure for “total chlorine” (i.e., active chlorine) is identified in that publication as Method 8167 appearing on page 379, Briefly, the “total chlorine” test involves introducing to the dilute water sample containing active halogen, a powder comprising DPD indicator powder, (i.e., N,N′-diethyldiphenylenediamine), KI, and a buffer. The active halogen species present react(s) with KI to yield iodine species which turn the DPD indicator to red/pink. The intensity of the coloration depends upon the concentration of “total chlorine” species (i.e., active chlorine”) present in the sample. This intensity is measured by a colorimeter calibrated to transform the intensity reading into a “total chlorine” value in terms of mg/L Cl₂. If the active halogen present is active bromine, the result in terms of mg/L Cl₂ is divided by 2.25 to express the result in terms of mg/L Br₂ of active bromine.

In greater detail, the DPD test procedure is as follows:
1. To determine the amount of species present in the water which respond to the “total chlorine” test, the water sample should be analyzed within a few minutes of being taken, and preferably immediately upon being taken.
2. Hach Method 8167 for testing the amount of species present in the water sample which respond to the “total chlorine” test involves use of the Hach Model DR 2010 colorimeter. The stored program number for chlorine determinations is recalled by keying in “80” on the keyboard, followed by setting the absorbance wavelength to 530 nm by rotating the dial on the side of the instrument. Two identical sample cells are filled to the 10 mL mark with the water under investigation. One of the cells is arbitrarily chosen to be the blank. To the second cell, the contents of a DPD Total Chlorine Powder Pillow are added. This is shaken for 10-20 seconds to mix, as the development of a pink-red color indicates the presence of species in the water which respond positively to the DPD “total chlorine” test reagent On the keypad, the SHIFT TIMER keys are depressed to commence a three minute reaction time. After three minutes the instrument beeps to signal the reaction is complete. Using the 10 mL cell riser, the blank sample cell is admitted to the sample compartment of the Hach Model DR 2010, and the shield is closed to prevent stray light effects. Then the ZERO key is depressed. After a few seconds, the display registers 0.00 mg/L Cl₂. Then, the blank sample cell used to zero the instrument is removed from the cell compartment of the Hach Model DR 2010 and replaced with the test sample to which the DPD “total chlorine” test reagent was added. The light shield is then closed as was done for the blank, and the READ key is depressed. The result, in mg/L Cl₂ is shown on the display within a few seconds. This is the “total chlorine” level of the water sample under investigation.

In the practice of this invention the microbiocidal system can be used in various ways. For example, a microbiocidally effective amount of a microbiocide used in this invention, preferably a bromine-based microbiocidal composition of type A) above and more preferably of type B) above, can be applied to the teats and preferably also to the surrounding udder area while in the form of a ordinary aqueous solution or spray, in the form of a thickened or gelled solution, in the form of a liquid film-forming composition, or in the form of a foam. In forming these compositions, various supplemental components or ingredients can be included in addition to the antimicrobial agent(s) used in the practice of this invention.

In the compositions of this invention at least one rheology modifier, or at least one organic water-soluble film-forming agent, or at least one emollient, or a combination of any two types or all three types of these components are present. Other ingredients can also be used if desired.

Pseudo plastic aqueous rheology can be effected in the compositions of this invention by inclusion of one or more rheology modifiers. Materials of this include polymeric materials such as a xanthan gum and polyvinyl alcohol compositions. When shear stress is applied to the composition (i.e., dipping), product viscosity is reduced allowing easy and rapid application to the teat; and, upon the release of shear (i.e., removal of source), total viscosity recovery occurs almost instantaneously immobilizing the coating, providing cling and assuring little waste by drippage. Further, such compositions have little or no viscoelastic character which thus allows the antimicrobial composition to flow and to coat the teat smoothly, forming a continuous layer over the skin of the teat without formation of muscilage streamers as the applicator is withdrawn. The compositions tend to flow slightly down the teat following application to form a thicker layer or “plug” across the orifice of the teat canal; and, thus cause a more effective prophylactic barrier against bacteria entering the teat canal.

The enhanced viscosity, thickening, or clinging action provided by the rheology modifier enables the composition to remain in contact with transient and resident pathogenic bacteria for longer periods of time, promoting microbiological efficacy and resisting waste because of excessive dripping. The rheology modifier may be a film former or act cooperatively with a film-forming agent to form a barrier that provides additional protection. Water soluble or water dispersible rheology modifiers that are useful can be classified as inorganic or organic. The organic thickeners can further be divided into natural and synthetic polymers with the latter still further subdivided into synthetic natural-based and synthetic petroleum-based.

Inorganic thickeners are generally compounds such as colloidal magnesium aluminum silicate, colloidal clays (e.g., bentonites), or silicas which have been fumed or precipitated to create particles with large surface to size ratios. Natural hydrogel thickeners of use are primarily vegetable derived exudates. For example, tragacanth, karaya, and acacia gums; and extractives such as caragheenan, locust bean gum, guar gum and pectin; or, pure culture fermentation products such as xanthan gum are all potentially useful in this invention. Chemically, all of these materials are salts of complex anionic polysaccharides. Synthetic natural-based thickeners having application are cellulosic derivatives wherein the free hydroxyl groups on the linear anhydro-glucose polymers have been etherified or esterified to give a family of substances which dissolve in water and give viscous solutions. This group of materials includes the alky- and hydroxyalkylcelluloses, specifically methylcellulose, hydroxyethyhmethylcellulose, hydroxypropylmethylcellulose, hydroxybutylmethylcellulose, hydroxyethylcellulose, ethylhydroxyethylcellulose, hydroxypropylcellulose, and carboxymethylcellulose. Synthetic petroleum-based water soluble polymers are prepared by direct polymerization of suitable monomers of which polyvinylpyrrolidone, polyvinylmethylether, polyacrylic acid and polymethacrylic acid, polyacrylamide, polyethylene oxide, and polyethyleneimine are representative.

All thickeners do not exhibit equal effectiveness in the compositions of this invention. Preferred aqueous thickening agents are those which are extremely pseudoplastic (non-Newtonian, rapid relaxation), tend not to develop a rigid three-dimensional structure from interpolymer interactions, have a low or negligible viscoelastic character and possess a high gel strength. Such rheological properties are manifested in a teat dip composition which has a smooth flowing appearance; is easy to pour and apply onto the teat, coats uniformly without forming muscilage streamers as the applicator is withdrawn and remains firmly inplace without significant sag. Examples of preferred rheology modifiers are xanthan gum and the hydroxylalkylcelluloses. Generally, the concentration of thickener used in the present invention will be dictated by the final composition any by the method of teat application. Spraying or misting requires a lower composition viscosity for easy and effective application of treatment than dipping. Film forming barrier dips typically require high apparent viscosity necessary to form thick coatings on teats which insures improved prophylactic effect.

Rheology modifier(s) when used in the compositions of this invention are typically used in proportions of up to about 10 wt % of the overall composition. Preferred proportions are in the range of about 0.01 to about 7.5 wt %, and particularly preferred proportions are in the range of about 0.1 to about 5 wt %, these proportions also being based on the total weight of the composition. It is desirable that the composition have the consistency of a relatively thick, hand lotion.

One or more water-soluble polymeric film forming agents can constitute another type of component that can be used in the compositions of this invention. These agents are typically form occlusive polymeric films or barriers that can be washed away from the teats and surrounding udder areas prior to milking by use of water, which in some cases should be warm water. Various materials are suitable for use as such film-formers in the compositions of this invention. Intermediate or fully hydrolyzed polyvinyl alcohol contribute to the mastitis control treatment, after drying, a balanced barrier layer which remains pliable and maintains integrity on the teat. In addition, the film may itself be rendered antimicrobial by envelopment of biocidal agents used in the practice of this invention. The film does not cause irritation and can provide significantly improved and prolonged protection to the teat during the intermilking period by structured adherence, yet does not sacrifice ease of removal prior to milking. Variation of film flexibility, water sensitivity, ease of solvation, viscosity, film strength and adhesion can be varied by adjusting molecular weight and degree of hydrolysis of the polyvinyl alcohol The preferred polyvinyl alcohol for use in compositions of this invention has a degree of hydrolysis greater than 92%, preferably greater than 98%, most preferably greater than 98.5%; and, has a molecular weight (Mn) that falls in the range of between about 15,000 and 100,000, and preferably between 40,000 and70,000 corresponding to a solution viscosity (4 wt % aqueous solution measured in centipoise (cP) at 20° C. by Hoeppler falling ball method) of 12-55 cP and 12-25 cP respectively.

Such intermediate or fully hydrolyzed polyvinyl alcohol film-forming agents when used in the compositions of this invention are typically used in proportions of up to about 12 wt % ofthe overall composition. Preferred proportions are in the range of about 0.01 to about 8 wt %, and particularly preferred proportions are in the range of about 0.1 to about 4 wt %, these proportions also being based on the total weight of the composition.

Also useful as a film-forming agent is a partially hydrolyzed grade of polyvinyl alcohol, i.e., a polyvinyl alcohol containing at least about 2 mole % residual vinyl acetate units. Aqueous film-forming coating compositions made using such materials can be applied in essentially the same manner as other water based teat sealers. These film-forming materials provide coatings or sealing compositions that are easily removable with a warm water rinse. Yet the coatings are indicated to be durable enough to be resistant to premature loss under a variety of actual field conditions, including complete immersion in water. Typically such film-forming agent is used in an amount in the range of more than 1 wt % but less than about 16 wt % of the total weight of the composition.

Other suitable film-forming agents useful in the compositions of this invention include hydroxyethylcellulose, methyl hydroxypropylcellulose, and ethylhydroxyethylcellulose. Many such film-forming agents are available on the open market as non-toxic, food grade materials. Chemically such products include nonionic water-soluble hydroxyethylcellulose, methyl hydroxypropylcellulose made from cellulose and propylene oxide, and non-ionic water soluble ethyl hydroxyethylcellulose. Such film-forming agents as supplied by the manufacturers can be used at a concentration in the range of about 0.25 to about 10 wt %, and preferably in the range of about 0.25 to about 6.0 wt% of the total weight of the composition. These film-forming agents provide a film that persists between milkings, yet can be readily removed before milking by typical pre-milling udder preparation such as washing with water or an aqueous sanitizer or by dipping the teat in a predip solution and wiping with a cloth or paper towel.

When employing a film forming agent, it is preferred to include in the composition an opacifying amount (preferably not more than about 10 wt %) of an opacifying agent, and it is particularly preferred to include a coloring agent (such as a food grade dye) in the aqueous composition. The use of such colorant makes it easier to see whether the coating has been completely removed during the washing prior to milking, especially if the color is blue or some other color that contrasts sharply from the color of the animal's skin.

Another component which can be used in the compositions of this invention is an emollient or humectant. Such substances lubricate, condition, and generally reduce and promote the healing of chapping or other types of skin irritation on the teat and surrounding surfaces which may result from environmental conditions such as wind chill, dehydration, abrasion and sunburn, or irritation caused during the course of the overall milking procedure. Any water-soluble or dispersible skin conditioning agent may be used in the compositions of this invention. Suitable substances which, if used in the compositions of this invention, serve as emollients or humectants include polyhydric alcohols such as glycerin, sorbitol, mannitol, and propylene glycol and its homopolymers; fatty acid esters of simple monohydric alcohols including isopropyl palmitate or isopropyl myristate and similar esters; polyol esters of fatty acids; and ethoxylated lanolins, vegetable oils, and similar natural-sourced derivatives such as aloe. The amounts of one or more emollients or humectants which may be included in the compositions of this invention can vary widely depending upon the consistency desired in the overall composition. Thus amounts typically in the range of up to about 60 wt %, and preferably in the range of about 1 to about 40 wt %, based on the total composition may be used. Amounts between about 0 to 20 wt % of the composition are often more preferred.

A variety of other components may be included in the compositions of this invention. One or more surfactants to provide emulsification or foaming action are one such type of useful but optional component. The surfactant(s) which may be included in the skin sanitizing compositions of this invention may be selected from a wide variety of materials including anionic, cationic, and non-ionic surfactants provided that the surfactant or surface active agent does not substantially deactivate the microbiocidal ingredient(s). Typical and suitable emulsifiers are the anionic surfactants which include the sulfonated detergents which comprises sulfonated fatty acids or sulfonated aliphatic hydrocarbon residues. A wide variety of sulfonated detergent surfactants are available for such use. Specific suitable anionic surfactants include sodium lauryl sulfate, sodium lauryl sarcosinate and sodium dodecyl benzenesulfonate, and similar substances.

Cationic surfactants are equally suitable for use in the skin sanitizing composition of this invention and illustrative examples of these surfactants include dimethylammonium chloride and cetyl trimethylammoniumchloride both of which are commonly used cationic surfactants or detergents. Alternatively, various non-ionic surfactants such as n-alkyl (C₁₂-C₁₆) dimethylammoniumoxide may also be employed in the preparation of the skin sanitizing composition of this invention.

Typical amounts of surfactant which may be used in preparing the skin sanitizing composition of this invention are in the range of about 0.5 to about 6 wt % of the total composition.

Alpha-hydroxycarboxylic acids, such as used in personal care products, can also be used in preparing the compositions of this invention. At use levels under 10 wt %, skin care benefits are indicated through a continued pattern of product usage.

Solubilizing agents a.k.a hydrotropes or couplers may also be used in the compositions of this invention to maintain physical single phase integrity and storage stability. To this end, any number of ingredients known to those skilled in formulation art may be employed, such as monofunctional and polyfunctional alcohols. These preferably contain from about 1 to about 6 carbon atoms and from 1 to about 6 hydroxy groups. Examples include ethanol, isopropanol, n-propanol, 1,2-propanediol, 1,2-butanediol, 2-methyl-2,4-pentanediol, mannitol and glucose. Also useful are the higher glycols, polyglycols, polyoxides, glycol ethers and propylene glycol ethers. Additional useful hydrotropes include the free acids and alkali metal salts of sulfonated alkylaryls such as toluene, xylene, cumene and phenol or phenol ether or diphenyl ether sulfonates; alkyl and dialkyl naphthalene sulfonates and alkoxylated derivatives. 1-Octane sulfonate or mixtures of 1-octane sulfonate and 1,2-octane disulfonate have also been recommended for use as hydrotropes.

Various other ingredients may be included in the skin sanitizing compositions of this invention if desired. These include, for example, inhibitors or stabilizers to provide shelf stability or similar functions, buffers to maintain pH control, sunscreen additives to protect against exposure to strong sunlight, foam stabilizers to enhance the consistency and duration of the composition when applied in the form of foam, chelating agents to increase cell wall permeability of mastitis-causing pathogens, and other antimicrobial additives.

The compositions of this invention for application to the animal will typically have a pH in the range of about 5 to about 9, and preferably in the range of about 6 to about 8.

In selecting components for use in the skin sanitizing compositions of this invention, care should be taken to ensure that the component is non-toxic at the levels employed, suitably compatible with the other components present, and that it does not impair the antimicrobial effectiveness of the resultant composition to any appreciable extent.

Aqueous compositions of this invention adapted for use in dry or non-lactating cow therapy may comprise in addition to type A), B), or C) component(s) referred to above, a film-forming polymer blend of a thermoplastic polyurethane having no reactive isocyanate groups and a hydrophilic poly(N-vinyllactam). Such film forming blends and the use are described in U.S. Pat. No. 6,440,442.

Use of film-forming polymers for application to bovine teats is described for example in U.S. Pat. Nos. 5,017,369 and 5,776,479, and such procedures can be utilized by employing the antimicrobial agents of this invention in lieu of (or in addition to) those described in these patents.

Procedures and apparatus for generating and using germicidal bovine teat dips or washes in the form of foams are described for example in U.S. Pat Nos. 6,302,058 and 6,348,206, and such procedures can be utilized by employing the antimicrobial agents of this invention in lieu of (or in addition to) those described in these patents.

The following Example illustrates the practice and some of the advantages of this invention. It is not intended that this Example shall in any way impose a limitation on the scope of this invention.

### EXAMPLE

A comparative study was conducted to determine the efficacy of the a composition used pursuant to this invention on bacteria when used as a post-dip for dairy cattle after milking. The biocidal agent employed was a sulfamate-stabilized active bromine composition available under the trademark STABROM® 909 biocide. This biocide was applied to the teats of cattle immediately after milking. After application to the teats tested, each teat (test AND control) had a surface sample taken to test for the presence of bacteria (mainly Staphylococcus aureus, Streptococcus agalactiae, Escherichia coli, Kiebsiella ssp., and Streptococcus uberis). For comparative purposes a commercially-available biocide for this use (UDDER GOLD® biocide (Alcide Corporation, Redmond, Wash.), was also tested.

The test procedure used was as follows:
a) Pre-Examination: All teats from all cows to be tested were examined for injuries before initiating test procedures. Any cows with injured, abnormal, or deformed teats were be excluded from testing to ensure uniformity.
b) Preparation: Each teat (test AND control) was fore-stripped prior to application of test material and after milking.
c) Application: A solution of STABROM 909 biocide and water was premixed with an inclusion rate of 30 mL of STABROM 909 biocide concentrate to one (1) liter of water. The solution was applied inmediately after fore-stripping to two (2) diagonal teats on each cow using standard post-dip procedures. The application was applied using a minimum standard of 15-30 seconds application time. After application, the teats were dried using service paper towels.
d) Sample Collection: Samples were collected for each teat on each cow using a cup (˜2.5 inches tall and 1.5 inches in diameter) filled with ˜1 inch of water after 30 seconds post-application. After application of solution to the teat and drying of the teat with a paper towel, the cup was placed onto the teat to get samples of microorganisms on the teat. This step took place within 15-30 seconds after application of test material, in order for normal post-milking procedures to occur. Each sample cup was labeled using numbers (e.g., Cow number 1) and description of either test or control (using “T” for test or “C” for control).
e) Number of Samples for Collection: A total of 24 cows were used for this trial. The site uses and 8×8 parlor. A total of 3 parlor cycles were tested, more if any cows are found with injured, abnormal, or deformed teats. A total of 96 bacteria samples were collected (4 per cow) from each teat of each cow.
f) Bacteria Testing: A sample of 0.01 ml were streaked on trypticase soy agar (TSA) containing 5% bovine calf blood. Samples were incubated at 37° C. for 48 hours, and then examined to identify microorganisms present. An accurate count of CFU's (Colony Forming Units) were recorded. The treatment group was compared to the control group for differences in bacteria counts.

A total of 24 dairy cows was used. Each cow served as it's own internal control with 3 of the 4 available teats serving as treatments as indicated in Table 1.

TABLE 1

| | Test Material | | Teats per |
|---|---|---|---|
| Test Group | and Inclusion Level | Reps¹ | Cow Used |
| Group 1 | Control (no disinfectant addition) | 24 | 1 |
| Group 2 | STABROM 909 biocide (30 mL per | 24 | 1 |
| | liter dilution) | | |
| Group 3 | UDDER GOLD biocide | 24 | 1 |

¹Each cow is used as it's own control.

Table 2 summarizes the results of these tests.

TABLE 2

Treatment Type (% change
after 30 sec from control)

| | STABROM 909 | UDDER GOLD |
|---|---|---|
| Bacteria Species | biocide | biocide |
| Staphylococcus aureus | 99.9999 | 99.9999 |
| Streptococcus agalactiae | 99.9999 | 99.9999 |
| Escherichia coli | 99.9999 | 98 |
| Klebsiella ssp. | 98 | 99.9999 |
| Streptococcus uberis | 99.9999 | 99.9999 |

Compounds referred to by chemical name or formula anywhere in this document, whether referred to in the singular or plural, are identified as they exist prior to coming into contact with another substance referred to by chemical name or chemical type (e.g., another component, a solvent, or etc.). It matters not what chemical changes, if any, take place in the resulting mixture or solution, as such changes are the natural result of bringing the specified substances together under the conditions called for pursuant to this disclosure.

As indicated above the term “derivative product” refers to the species of biocide that form and exist in the aqueous medium upon adding the biocidal compound or composition to the aqueous medium. In short, the “derivative product” is whatever forms when the biocidal compound or composition is dissolved in an aqueous medium.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

All documents referred to herein are incorporated herein by reference in toto as if fully set forth in this document.

This invention is susceptible to considerable variation in its practice.

1. A method of preventing or controlling bovine mastitis, which method comprises treating at least teats of the animal with an effective antimicrobial amount of:
A) a composition comprised of an aqueous microbiocidal solution of one or more active halogen species, which solution is a derivative product in an aqueous medium of (i) bromine, chlorine, and bromine chloride, or any two or all three thereof, and (ii) a water-soluble source of sulfamate anion; or
B) a composition comprised of an aqueous microbiocidal solution of one or more active halogen species, which solution is a derivative product in an aqueous medium of at least one 1,3-dihalo-5,5-dialkylhydantoin in which one of the halogen atoms is a bromine atom and the other halogen atom is a chlorine or bromine atom, and in which when both halogen atoms are bromine atoms, one of the alkyl groups is a methyl group and the other alkyl group contains in the range of 1 to about 4 carbon atoms, and when one of the halogen atoms is a bromine atom and the other halogen atom is a chlorine atom, the alkyl groups, independently, each contain in the range of 1 to about 4 carbon atoms; or
C) a composition comprised of an aqueous microbiocidal solution of A) and an aqueous microbiocidal solution of B).

2. A method as in claim 1 wherein the composition is applied in the form of a teat dip, as a wash, or in the form of a spray.

3. A method as in claim 1 wherein the composition is applied in the form of a foam.

4. A method as in any of claims 1-3 wherein the composition further comprises:
D) at least one thickener, or
E) at least one water-soluble polymeric film-forming agent; or
F) at least one emollient or humectant.

5. A method as in claim 4 wherein the composition comprises at least two of D), E), and F).

6. A method as in claim 4 wherein the composition comprises each of D), B), and F).

7. A method as in any of claims 1-6 wherein the treating is carried out using a composition of A).

8. A method as in claim 7 wherein the composition of A) is formed by a reaction in water between (i) bromine, (ii) bromine chloride, (iii) bromine and chlorine where the molar amount of bromine exceeds the molar amount of chlorine, or (iv) a mixture of any two or all three of (i), (ii), and (iii), and a water-soluble source of sulfamate.

9. A method as in claim 8 wherein the reaction is performed with the water at a pH of at least about 10.

10. A method as in claim 9 wherein the reaction is performed using bromine chloride or a mixture of bromine chloride and bromine, wherein the sulfamate source is an alkali metal sulfamate, and wherein the pH is maintained by use of a water-soluble sodium or potassium base.

11. A method as in any of claims 1-6 wherein the treating is carried out using a composition of B).

12. A method as in claim 11 wherein the solution of B) is formed from at least one N,N′-bromochloro-5,5-dialkylhydantoin in which the alkyl groups, independently, each contain in the range of 1 to about 4 carbon atoms.

13. A method as in claim 11 wherein the solution of B) is formed from N,N-bromochloro-5,5-dimethylhydantoin.

14. A method as in claim 11 wherein the solution of B) is formed from at least one 1,3-dibromo-5,5-alklylhydantoin in which one of the alkyl groups is a methyl group and the other alkyl group contains in the range of 1 to about 4 carbon atoms.

15. A method as in claim 11 wherein the solution of B) is formed from 1,3-dibromo-5,5-dialkylhydantoin.

16. A composition adapted for preventing or controlling bovine mastitis, which composition comprises:
A) an aqueous microbiocidal solution of one or more active halogen species, which solution is a derivative product in an aqueous medium of (i) bromine, chlorine, and bromine chloride, or any two or all three thereof, and (ii) a water-soluble source of sulfamate anion; or
B) an aqueous microbiocidal solution of one or more active halogen species, which solution is a derivative product in an aqueous medium of at least one 1,3-dihalo-5,5-dialkylhydantoin in which one of the halogen atoms is a bromine atom and the other halogen atom is a chlorine or bromine atom, and in which when both halogen atoms are bromine atoms, one of the alkyl groups is a methyl group and the other alkyl group contains in the range of 1 to about 4 carbon atoms, and when one of the halogen atoms is a bromine atom and the other halogen atom is a chlorine atom, the alkyl groups, independently, each contain in the range of 1 to about 4 carbon atoms; or
C) an aqueous microbiocidal solution of A) and B);
wherein the composition additionally contains at least one of the following components:
D) at least one thickener, or
E) at least one water-soluble polymeric film-forming agent; or
F) at least one emollient or humectant.

17. A composition as in claim 16 wherein the composition is in the form of a dip, wash, spray, or foam.

18. A composition as in either of claims 16 or 17 wherein the composition comprises at least two of D), E), and F).

19. A composition as in either of claims 16 or 17 wherein the composition comprises each of D), E), and F).

20. A composition as in any of claims 16-19 the aqueous microbiocidal solution of the composition is a solution of A).

21. A composition as in claim 20 wherein the solution of A) is formed by a reaction in water between a water-soluble source of sulfamate and (i) bromine, (ii) bromine chloride, (iii) bromine and chlorine where the molar amount of bromine exceeds the molar amount of chlorine, or (iv) a mixture of any two or all three of (i), (ii), and (iii).

22. A composition as in claim 21 wherein the reaction is performed with the water at a pH of at least about 10.

23. A composition as in claim 22 wherein the reaction is performed using bromine chloride or a mixture of bromine chloride and bromine, wherein the sulfamate source is an alkali metal sulfamate, and wherein the pH is maintained by use of a water-soluble sodium or potassium base.

24. A composition as in any of claims 16-19 wherein the aqueous microbiocidal solution of the composition is a solution of B).

25. A composition as in claim 24 wherein the solution of B) is formed from at least one N,N′-bromochloro-5,5-dialkylhydantoin in which the alkyl groups, independently, each contain in the range of 1 to about 4 carbon atoms.

26. A composition as in claim 25 wherein the solution of B) is formed from N,N′-bromochloro-5,5-dimethylhydantoin.

27. A composition as in claim 24 wherein the solution of B) is formed from at least one 1,3bromo-5,5-dialkylhydantoin in which one of the alkyl groups is a methyl group and the other alkyl group contains in the range of 1 to about 4 carbon atoms.

28. A composition as in claim 27 wherein the solution of B) is formed from 1,3-dibromo-5,5-dialkylhydantoin.

29. A method of preventing or controlling bovine mastitis, which method comprises
1) applying at least to teats of the animal, an effective antimicrobial amount of a composition as in any of claims 16-28 having a pH in the range of about 6 to about 9; and
2) after a non-irritating, non-harmful contact time of not more than about 3 minutes, washing at least the areas of the animal to which the composition was applied so as to remove the applied antimicrobial amount of the composition from said areas.

30. A method as in claim 29 wherein said pH is in the range of about 6 to about 8 and wherein said contact time is not more than 2 minutes.

31. A method as in either of claims 29 or 30 wherein said contact time is not more than about 15 seconds.
